(19) United States
(12) Patent Application Publication
(10) Pub. No.: US 2016/0151530 A1
(43) Pub. Date: Jun. 2, 2016

(54) Sanitization Device Using Electrical Discharge

(71) Applicant: HITACHI, LTD., Tokyo (JP)

(72) Inventors: Tetsuo KAWANABE, Tokyo (JP); Takumi TANDOU, Tokyo (JP); Masanori AKIMOTO, Tokyo (JP)

(73) Assignee: Hitachi, Ltd., Chiyoda-ku, Tokyo (JP)

(21) Appl. No.: 14/906,001

(22) PCT Filed: Jul. 31, 2013
(86) PCT No.: PCT/JP2013/070693

§ 371 Date: Jan. 19, 2016

(51) Int. Cl. A61L 9/22

(52) U.S. Cl. CPC A61L 9/22

(57) ABSTRACT

A sterilization device includes: a charged fine water droplet supplying unit; and a plasma generating unit, wherein the charged fine water droplet supplying unit and the plasma generating unit are provided on a wall surface of a wind channel in order of the charged fine water droplet supplying unit and the plasma generating unit from an upper stream in a direction in which air flows, the plasma generating unit includes a charged fine water droplet supplying part and a plasma generator, the charged fine water droplet supplying part includes a high-voltage power supply, an earthed electrode, and an electrode to which moisture is supplied by a water feeding unit, a negative high voltage with respect to the earthed electrode being applied to the electrode supplied with moisture, the plasma generator includes a pair of plasma generating electrodes and a high-frequency power supply, the plasma generating electrodes being covered by a dielectric substance and being provided in the same plane with the dielectric substance, and the air is converted into plasma and emitted when voltage is applied to the plasma generating electrodes by the high-frequency power supply. Thus, it is possible to make a strong sterilization effect and control of a toxic substance compatible.

### TECHNICAL FIELD

The present invention relates to a sterilization device of an airborne bacterium.

### BACKGROUND ART

Recently, there is an increasing need for a sterilization technology of an airborne bacterium for an aseptic room in medical and biotechnology fields or for sterilization or deodorization in an indoor space in a general household. Specifically, in the medical and biotechnology fields, a new sterilization device which has a high sterilization effect and which can secure safety for a human body is desired. As conventional representative sterilization technologies, there are filtering sterilization, sterilization by an ultraviolet ray or radial ray, and sterilization by gas, for example. The filtering sterilization is a method to remove a microorganism by filtering air with an HEPA filter or the like. The sterilization by an ultraviolet ray or radial ray is a method to perform sterilization by emitting an ultraviolet ray or radial ray to a microorganism and to deteriorate its DNA or cell wall. The sterilization by gas is a method to perform sterilization by filing a room with toxic gas such as an ethylene oxide gas or formaldehyde gas.

The above-described general sterilization technologies are practically used in various fields. However, further consideration is necessary to make a high sterilization effect and safety for a human body compatible. For example, the filtering sterilization does no harm to a human body. However, there is a problem that a microorganism smaller than a pore size of a filter cannot be removed. Also, since a filter itself does not have a sterilization effect, there is a concern that a microorganism which is trapped once may be scattered in the air again. A low stabilization property is a problem of the sterilization by an ultraviolet ray. It is necessary to emit the ultraviolet ray for a long period in order to acquire a high sterilization effect. In the sterilization by a radial ray, a large shield facility is necessary to prevent the radial ray from spreading into a space other than a space to be sterilized. Since toxic gas is used in the sterilization by gas, there is a problem that a degassing process after the sterilization processing takes a long period. Moreover, a risk in aspiration of toxic gas is a problem. On the other hand, a sterilization technology using electric discharge is focused as a method which is relatively safer than the other sterilization methods and which can secure a high sterilization effect. The sterilization using electric discharge is a method to perform sterilization by generating active species having an oxidation effect by a corona discharge, a streamer discharge, or the like. The sterilization using electric discharge comes to be used in home electric appliances for sterilization or deodorization of an indoor space.

### CITATION LIST

### Patent Literature

PTL 1: Japanese Patent Application Laid-Open No. 2006-101912

PTL 2: Japanese Patent Application Laid-Open No. 2012-120677

### SUMMARY OF INVENTION

### Technical Problem

As described above, a sterilization technology using electric discharge is a relatively safe sterilization technology. However, there is a problem that ozone, which is a toxic substance, is generated as a by-product. A small amount of ozone exists in a natural atmosphere. However, when its concentration becomes high, ozone is toxic to a human body. In the sterilization technology using electric discharge, when an output of electric discharge is increased to increase an amount of generation of active species effective for sterilization, an amount of generated ozone is increased at the same time. That is, there is a trade-off relationship between a sterilization effect and the amount of generated ozone. Thus, in a present condition, it is necessary to control a sterilization effect to reduce the amount of generated ozone to a degree which is not harmful to a human body.

As an example of sterilization using electric discharge, for example, there is a method of generating active species by using a corona discharge or streamer discharge or a method of generating a charged fine water droplet by electrostatic atomization by applying high voltage to moisture. In PTL 1, an active species releasing device to simultaneously use active species generated in a discharging device and a droplet generated in a droplet generating device is proposed. According to this method, it is possible to acquire a high sterilization effect by generating active species and a droplet simultaneously compared to a method of applying active species or droplet independently.

However, in the sterilization device of PTL 1, the discharging device to generate active species and the droplet generating device are independent from each other and the discharging device is in an upper stream of the droplet generating device or is provided in parallel therewith. Thus, it is difficult to cause a reaction of a fine water droplet with active species, decomposition of a fine water droplet by electric discharge, and the like because of a configuration of the device and it is not possible to cause a reaction of generating an OH radical having a high sterilization effect. Also, in PTL 1, a discharging method (such as streamer discharge or corona discharge) in which a metal electrode is included as a configuration of a discharging part and directly makes a contact with a plasma is disclosed. In this discharging method, there is a case where the electrode is sputtered by a plasma generated by electric discharge and is worn out or deteriorated. Moreover, the electrode may be rusted due to a contact with moisture in the air. Also, when discharging electrodes are configured to face each other in the sterilization device using electric discharge, it is necessary to flow the air, which is to be sterilized, in a narrow gap between the electrodes to generate electric discharge. Thus, conductance of a channel of the air becomes small and utilization for sending a large amount of air becomes difficult.

In PTL 2, a mechanism to emit a functional mist by a plasma discharge and a fine droplet generating mechanism is proposed. In PTL 2, it is possible to generate an OH radical by causing a reaction of a fine droplet in a discharging part. Thus, a high sterilization property can be acquired. However, in the fine droplet generating mechanism of PTL 2, a unit to generate an electrically-neutral fine water droplet by heated steam or the like is proposed. A method to negatively charge a droplet and to efficiently supply the droplet to a plasma region having positive potential by an electric effect is not disclosed. Thus, a large amount of supplied moisture does not react in the plasma region and passes through the discharging part, whereby the OH radical cannot be generated efficiently. Moreover, a diameter of a water droplet generated by heating is larger than a diameter of a water droplet generated by Rayleigh breakup due to charging. Thus, it becomes possible to break up all water droplets in the plasma.

### Solution to Problem

In order to solve the above problem, (1) a sterilization device of the present invention includes: a charged fine water droplet supplying unit; and a plasma generating unit, wherein the charged fine water droplet supplying unit and the plasma generating unit are provided on a wall surface of a wind channel in order of the charged fine water droplet supplying unit and the plasma generating unit from an upper stream in a direction in which air flows, the plasma generating unit includes a charged fine water droplet supplying part and a plasma generator, the charged fine water droplet supplying part includes a high-voltage power supply, an earthed electrode, and an electrode to which moisture is supplied by a water feeding unit, a negative high voltage with respect to the earthed electrode being applied to the electrode supplied with moisture, the plasma generator includes a pair of plasma generating electrodes and a high-frequency power supply, the plasma generating electrodes being covered by a dielectric substance and being provided in the same plane with the dielectric substance, and the air is converted into plasma and emitted when voltage is applied to the plasma generating electrodes by the high-frequency power supply.

Also, (2) a sterilization device of the present invention includes: a charged fine water droplet supplying unit; a plasma generating unit; and blower means, wherein the charged fine water droplet supplying unit and the plasma generating unit are provided on a wall surface of a wind channel in this order from an upper stream in a direction in which air supplied by the blower means is sent, the plasma generating unit includes a charged fine water droplet supplying part and a plasma generator, the charged fine water droplet supplying part includes a high-voltage power supply, an earthed electrode, and an electrode to which moisture is supplied by a water feeding unit, a negative high voltage with respect to the earthed electrode being applied to the electrode to which moisture is supplied, the plasma generator includes a pair of plasma generating electrodes and a high-frequency power supply, the plasma generating electrodes being covered by a dielectric substance and being provided in the same plane with the dielectric substance, and the air is converted into plasma and emitted when voltage is applied to the plasma generating electrodes by the high-frequency power supply.

In addition, in (1) or (2), an OH radical is generated by converting air, which includes a charged fine water droplet generated in the charged fine water droplet supplying part, into plasma and emitting the plasma with the plasma generating unit.

Moreover, in (1) or (2), a surrounding part of the plasma is covered by a dielectric substance.

Also, in (1) or (2), a cross-sectional, area of a channel of the air is decreased in a following stage of the plasma generating unit.

Furthermore, in (1) or (2), a channel of the air is connected to a second channel, a flowing direction of which is different from that of the channel, in a following stage of the plasma generating unit.

Note that the sterilization in the present invention can be also referred to as disinfection, eradication of bacteria, antisepsis, or deodorization.

### Advantageous Effects of Invention

By using a sterilization device of the present invention, it is possible to supply a charged fine water droplet generated in a charged fine water droplet supplying unit to a plasma generating unit in a downstream and to make a strong sterilization effect and control of a toxic substance compatible.

### BRIEF DESCRIPTION OF DRAWINGS

FIG. 1 is a configuration view of a sterilization device according to a first embodiment of the present invention.

FIG. 2 is a view illustrating a structure of a channel of air according to the first embodiment of the present invention.

FIG. 3 is a view illustrating a structure of a different channel of the air according to the first embodiment of the present invention.

FIG. 4 is a view illustrating a configuration of a plasma generating part according to a second embodiment of the present invention.

FIG. 5 is a graph illustrating an amount of generated ozone of when plasma generated in the plasma generating part according to the second embodiment of the present invention is sandwiched by dielectric substances or is not sandwiched thereby.

FIG. 6 is a configuration view of an entire air conditioner according to a third embodiment of the present invention.

FIG. 7 is a side cross-sectional view of an indoor equipment including a sterilization device according to a third embodiment of the present invention.

FIG. 8 is a top view of the indoor equipment including the sterilization device according to the third embodiment of the present invention.

FIG. 9 is a configuration view of the sterilization device according to the third embodiment of the present invention.

FIG. 10 is a schematic view of a self-propelled cleaner for a bioclean room which cleaner includes a sterilization device according to the fourth embodiment of the present invention.

FIG. 11(a) is a side view of the self-propelled cleaner including the sterilization device according to the fourth embodiment of the present invention.

FIG. 11(b) is a bottom view of the self-propelled cleaner including the sterilization device according to the fourth embodiment of the present invention.

### DESCRIPTION OF EMBODIMENTS

### First Embodiment

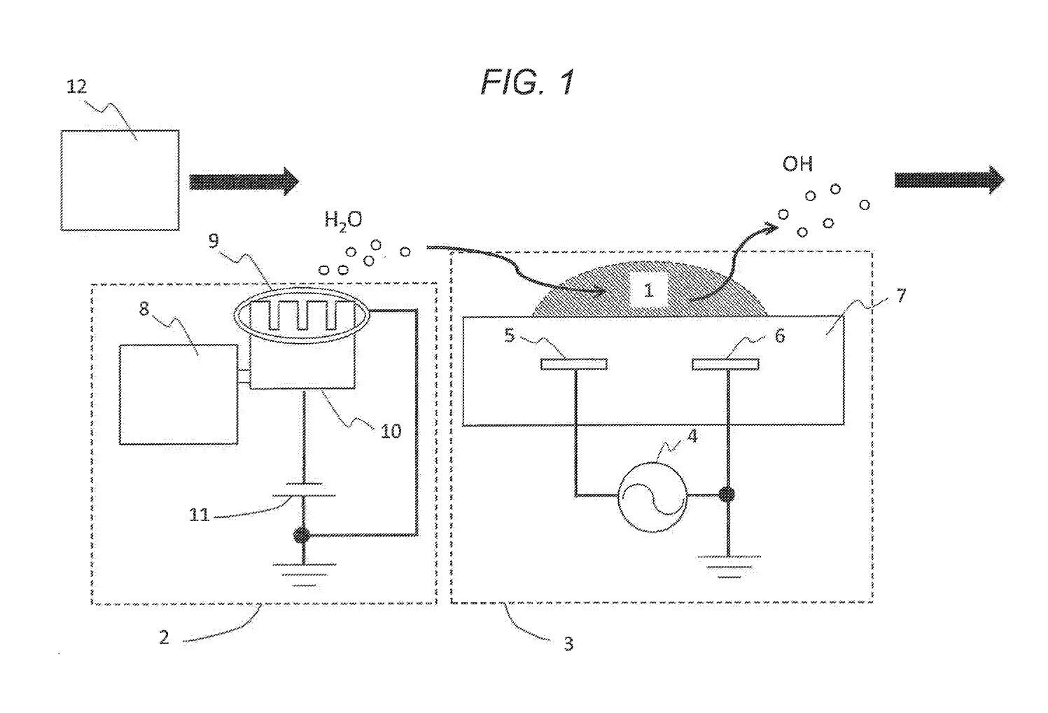

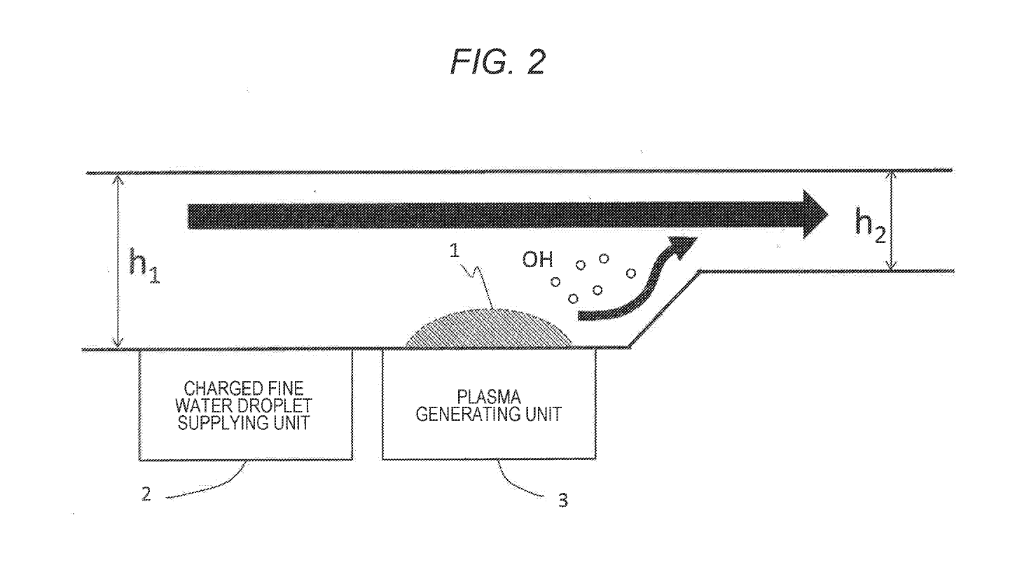

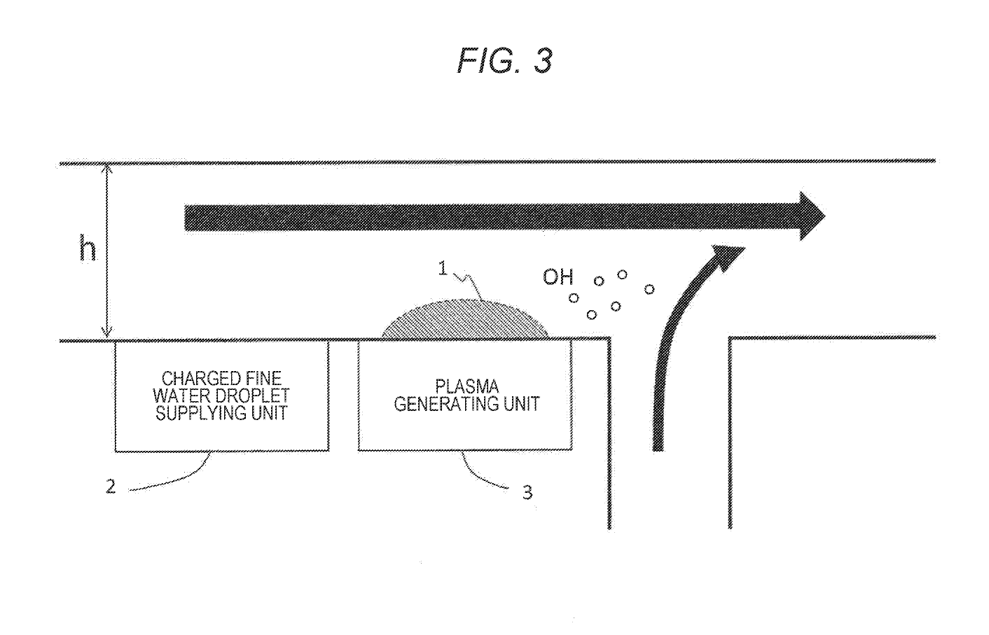

In the following, a first embodiment of the present invention will be described with reference to FIG. 1 to FIG. 3. FIG. 1 is a schematic view of a sterilization device of the present invention and each of FIG. 2 and FIG. 3 is a view illustrating an example of a structure of a channel of the present invention.

A sterilization device includes blower means 12, a charged fine water droplet supplying part 2, and a plasma generating part 3. The charged fine water droplet supplying part 2 and the plasma generating part 3 are provided in such a manner that air supplied by the blower means 12 passes through these parts in this order. The charged fine water droplet supplying part 2 includes an atomization electrode 10 to which moisture is supplied by a moisture supplying part 8, an earthed electrode 9 which is away from the atomization electrode 10 for 1 to 10 mm, and a high voltage DC power supply 11. A high voltage of −1 to −10 kV is applied to the atomization electrode 10 by the high voltage DC power supply 11 and electrostatic atomization of the moisture supplied to the atomization electrode 10 is performed. Moisture in a high electrical field breaks up into 10 to 50 nm by the Rayleigh breakup. A shape of the atomization electrode 10 preferably has a corner and an electric field concentrated part in such a manner that electrostatic atomization of moisture in the high electrical field can be performed easily. In order to supply the moisture from the moisture supplying part 8 to the high electrical field part in the atomization electrode 10, a material with hygroscopicity such as an acrylic fiber or a sponge is used for the atomization electrode 10. Also, a shape of the atomization electrode may be a needle-shape. In this case, a method to provide a fine channel in the atomization electrode 10 and to supply moisture to an electrode concentrated part by using a capillary action can be used.

The plasma generating part 3 includes an electrode 5 to which voltage is applied by a high-frequency power supply 4, an earthed electrode 6, and a dielectric substance 7 which covers surfaces of the electrode 5 and the electrode 6. The electrode 5, the electrode 6, and the dielectric substance 7 generate a plasma in a vicinity of a surface of the dielectric substance 7 by a surface discharging-type discharging method in which the three are provided in the same plane. As the dielectric substance 7, a dielectric substance with an ozone-catalyst effect and high plasma resistance such as Al₂O₃, or MnO₂ having a high ozone-catalyst effect is preferably used. A voltage of 300 V to 5 kV is applied to the electrode 5 at a frequency of 1 kHz to 100 kHz. The electrode 5 generates a high electrical field on the surface of the dielectric substance 7, causes breakdown of the air, and generates plasma 1. When a certain amount of electric charge is accumulated on the surface of the dielectric substance 7, the electric discharge is automatically stopped. Thus, a spark is not generated. Also, by a discharging method such as a streamer or corona discharge in which method plasma directly makes a contact with a metal electrode, the electrode may be worn out by plasma sputtering or may be rusted and performance thereof may be deteriorated due to the supplied moisture. However, a metal electrode is covered by a dielectric substance in the present embodiment. Thus, there is no such a concern.

In the charged fine water droplet supplying part 2, only a negatively-charged fine water droplet among charged fine water droplets generated in the atomization electrode 10 is emitted to a space by electric repulsive force from the atomization electrode 10 having negative potential. Since the negatively-charged fine water droplet is electrically attracted to plasma having positive potential, reaction in a plasma-generated region can be performed efficiently. Moreover, a diameter of the charged fine water droplet is 10 to 50 nm. Thus, the charged fine water droplet has an extremely small diameter and a large surface area with respect to the same volume compared to a water droplet having a diameter larger than 1 μm which droplet is generated by a moisture generating method such as steaming. Thus, it is easy to proceed a chemical reaction in the charged fine water droplet compared to steam or the like. Thus, even when the same amount of moisture added, an effect of reducing ozone and an effect of generating OH radical become high.

In a plasma generator 3, a charged fine water droplet generated in the charged fine water droplet supplying part 2 consumes ozone O₃, for example, by reactions expressed in reaction formulas (3), (5), and (6). Also, when an O atom generated in the plasma-generated region is consumed in the reaction with water, it becomes difficult to get an ozone generating reaction expressed by the reaction formula (1). According to the above effect, it is possible to reduce the amount of generated ozone by supplying the charged fine water droplet to the plasma generator 3. Also, by reactions expressed by the reaction formulas (2), (4), and (5), an OH radical with high oxidizability is generated. Since a reduction time constant of the OH radical is short and is 50 to 100 μs, the OH radical does not remain in the air. Thus, according to the present embodiment, only the passing air can be sterilized and safety for a human body can be secured.

O+O₂+M→O₃ (1)

e+H₂O→H+OH+e (2)

OH+O₃→O₂+HO₂ (3)

HO₂+O→O₂+OH (4)

HO₂+O₃→O₂+O₂+OH (5)

H₂O+O₃→OH+OH+O₂ (6)

Also, since plasma has positive potential (plasma potential) with respect to a wall surface, a negatively-charged fine water droplet is electrically attracted to the plasma and causes a reaction efficiently in the plasma-generated region. Thus, it is possible to increase an effect of reducing the amount of ozone and an effect of generating an OH radical. Moreover, since the charged fine water droplet is forcibly sent to the plasma generating part by the blower means, it is possible to increase an effect of adding moisture to the plasma.

With reference to FIG. 2, an example of a structure of a channel of the air which passes through a sterilization unit will be described. A reaction time constant of the OH radical is short and is 50 to 100 μs. Thus, only the air in the vicinity of the plasma can be sterilized. Thus, a channel (h1>h2) a cross-sectional area of which is smaller than that in an upper stage is provided in a following stage of a plasma generating unit 3. By providing a channel with a small cross-sectional area in a lower stream of the plasma generating unit, an OH radical generated in the vicinity of the plasma generator can be carried to a main stream by forced-convection and most of passing air can be sterilized. Alternatively, as illustrated in FIG. 3, by providing a second channel in a lower stream of the plasma generating unit and by supplying the air from the second channel, the OH radical generated in the vicinity of the plasma generator can be carried to the main stream.

Moreover, since the plasma generating method of the present embodiment is a surface discharging type, there is no limitation in a structure of a channel compared to a discharging method in which electrodes face each other. For example, it is possible to increase conductance of the passing air by increasing a size of a channel diameter h. That is, it is possible to flow a large amount of passing air and to perform sterilization at high speed.

Also, in the first embodiment, a case where an object of processing is an airborne bacterium has been described. However, a sterilization property can be acquired as long as the OH radical generated by the sterilization device makes a contact with the object of processing. Thus, when the sterilization device is brought close to a wall surface or blowing is performed by the blower means at high speed in order to prevent deactivation of the OH radical, the device is also effective to an adhered bacterium.

### Second Embodiment

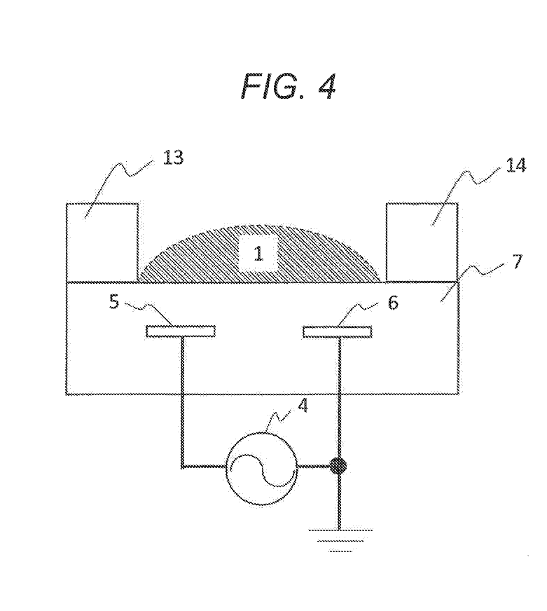

A preferable embodiment with which the amount of generated ozone can be reduced in the plasma generating part 3 in the configuration described in the first embodiment will be described with reference to FIG. 4.

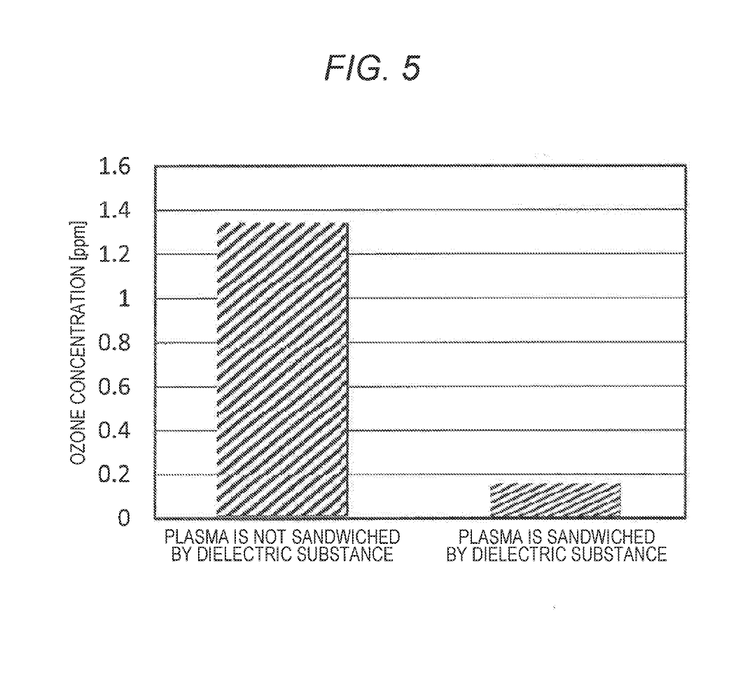

In a plasma generating part 3, dielectric substances 13 and 14 are provided to sandwich plasma 1. As the dielectric substances 13 and 14, Al₂O₃ or MnO₂ having an ozone catalytic reaction is preferably used. By providing the dielectric substances 13 and 14, an area in which the plasma 1 makes contact with a catalyst is increased and an ozonolysis reaction is promoted. In order to verify the above effect, an experiment to measure the amount of generated ozone of when Al₂O₃ is used as the dielectric substances 13 and 14 and in a case where a charged fine water droplet is not supplied is performed. A result of the experiment will be described with reference to FIG. 5. FIG. 5 is a view illustrating a result of measuring an ozone concentration in a 35 mm lower stream of the plasma generating part with an ozone concentration meter. When the plasma is not sandwiched by the dielectric substances, an ozone concentration is 1.35 ppm. On the other hand, when the dielectric substances are provided to sandwich the plasma, the concentration is 0.16 ppm and the amount of generated ozone can be reduced for 88%. As illustrated in the first embodiment, it is possible to reduce the amount of ozone by supplying a charged fine water droplet.

Moreover, it is possible to activate a catalyst by providing a heater in the dielectric substances 7, 13, and 14 and heating the three and to increase an ozone decomposition effect. Alternatively, BaTiO₃ with a high dielectric loss may be used for the dielectric substances 7, 13, and 14 instead of providing the heater. When a high frequency voltage is applied to a dielectric substance with a high dielectric loss such as BaTiO₃, dielectric heating is caused by a dielectric loss. Also, while a discharge starting voltage in a dielectric substance with a low-dielectric constant (such as Al₂O₃) is 1 kV or higher, it is possible to decrease a discharge starting voltage to 300 to 500 V in BaTiO₃ or the like with a high-dielectric constant. Alternatively, the surfaces of the dielectric substances 7, 13, and 14 may be heated with an infrared ray from the outside.

As described above, it is possible to dramatically reduce the amount of ozone generated by electric discharge and to further improve safety for a human body. In other words, it is possible to increase a sterilization property while keeping the safety even when discharge power is further increased.

### Third Embodiment

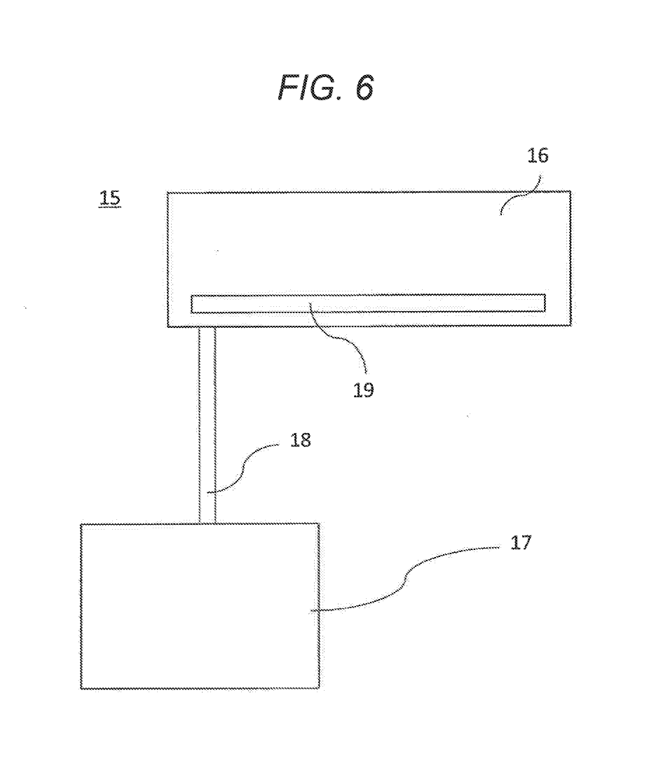

An embodiment in which a sterilization device having configurations described in the first and second embodiments is applied to an indoor equipment of an air conditioner will be described with reference to FIG. 6 to FIG. 8. An air conditioner makes the indoor air pass through a heat exchanger, converts the air into heated, cooled, or dehumidified air (conditioned air), and blows out this air into a room. The indoor air includes an unpleasant substance, which includes various smelling components, or a toxic substance such as mold, a virus, or a bacterium. It is desired to remove these substances.

FIG. 6 is a configuration view of an entire air conditioner 15 of the present embodiment. The air conditioner 15 includes an indoor equipment 16 and an outdoor equipment 17 and a connection pipe 18 through which a refrigerant passes connects therebetween. In the air conditioner 15, a refrigerant is circulated by a compressor in the outdoor equipment 17. In the outdoor equipment 17, the refrigerant absorbs/releases heat from/to the outdoor air, whereby temperature is adjusted. The indoor air passing through the indoor equipment 16 exchanges heat with the refrigerant. Then, the air is blows out from an air outlet 19 and air-conditions the room.

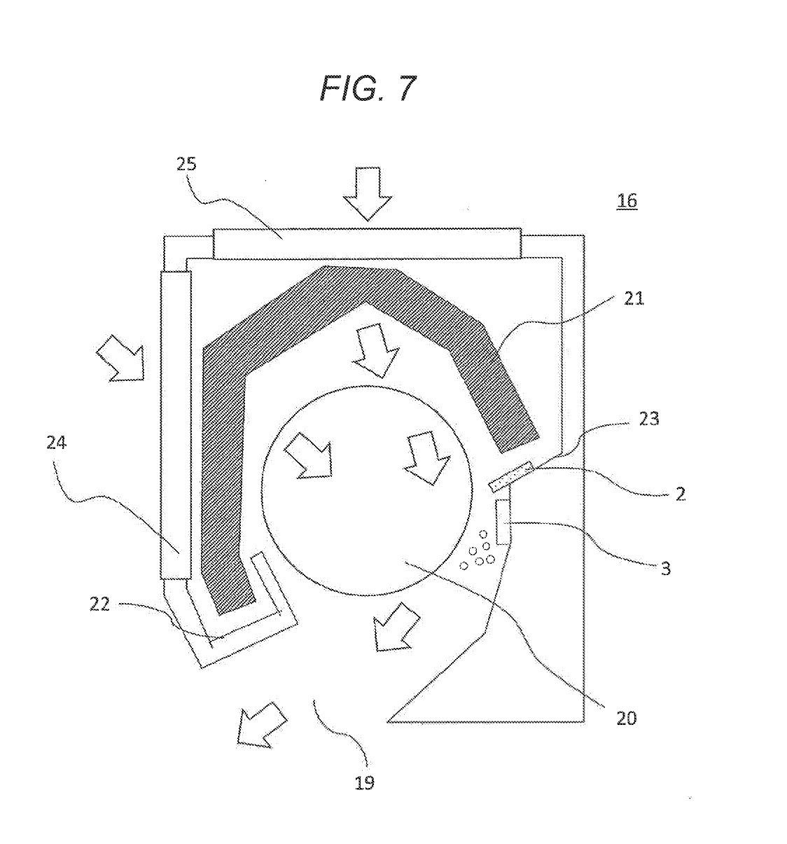

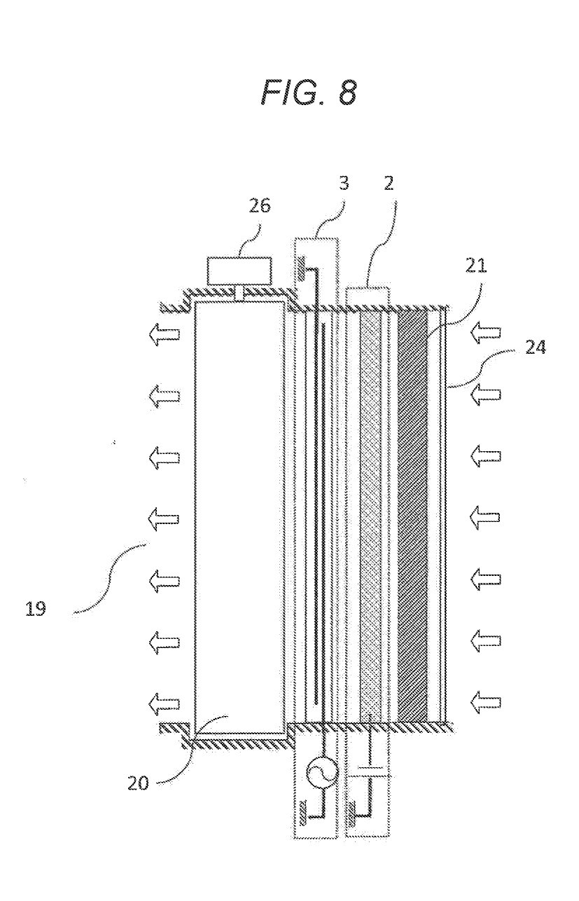

A basic structure inside the indoor equipment 16 and a sterilization device according to the present invention will be described with reference to FIG. 7 and FIG. 8. FIG. 7 is a side cross-sectional view of the indoor equipment 16 including the sterilization device according to the present invention and FIG. 8 is a top view thereof. In FIG. 7, a blower fan 20, a heat exchanger 21, and dew receiving trays 22 and 23 are attached to the inside of the indoor equipment 16. The heat exchanger 21 is arranged on an inlet side of the blower fan 20 and is formed in a substantially-inverse V-shape. In FIG. 8, the blower fan 20 is rotated by a fan motor 26 and the air is sent by many wings included in the blower fan 20. As a result, in each of FIG. 7 and FIG. 8, the air flows in a manner indicated by an outline arrow. The blower fan 20 is arranged at the center in the indoor equipment 16 in such a manner that the indoor air blows in from air inlets 24 and 25 and blows out from the air outlet 19. To the air outlet 19, an air deflector with which a direction of ending the air can be controlled is preferably provided and the air is preferably sent in an intended direction. To the air inlets 24 and 25, a filter is preferably provided to remove a dust included in the indoor air. In FIG. 7, the sterilization device according to the present invention is provided on a wall surface on the side of the blower fan 20 of the dew receiving tray 23.

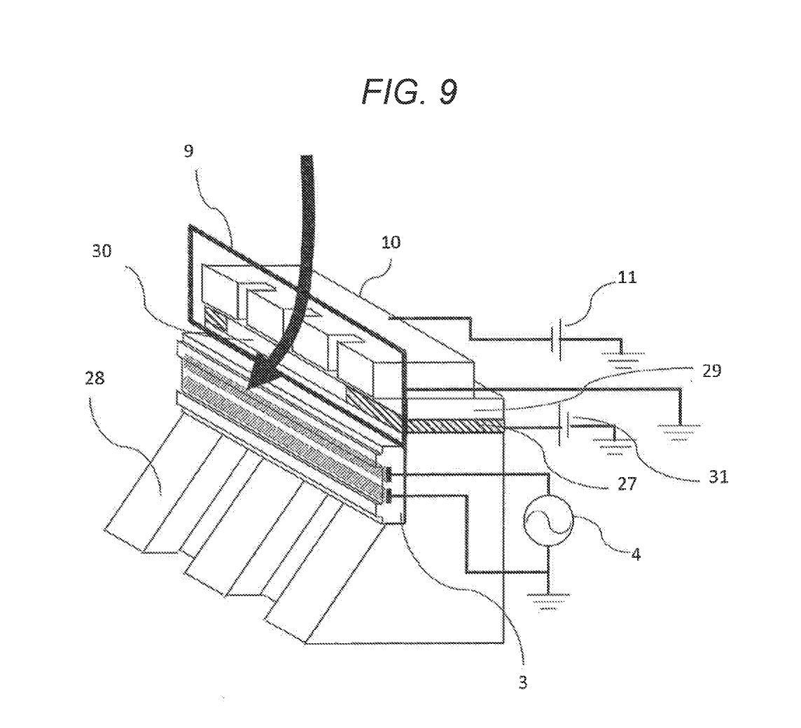

A detail of the sterilization device will be described with reference to FIG. 9. The sterilization device includes a charged fine water droplet supplying part 2 and a plasma generating part. Blower means of the sterilization device is the wind from the blower fan 20. By the blower fan 20, the wind passing through the indoor equipment 16 flows in a manner indicated by a block arrow. With the dew receiving tray 23, a Peltier element 27, a heat sink 28, and a cooling plate 29, moisture to be supplied to an atomization electrode 10 of the charged fine water droplet supplying part 2 is generated. A plurality of Peltier elements 27 is connected to a DC power supply 31 and is provided in series in the same plane. Between the Peltier elements 27, a heat insulation material 30 is provided. When power is applied to the Peltier elements 27, surfaces, which are on the side of the cooling plate 29, of the Peltier elements 27 are cooled and surfaces on the side of the heat sink 28 are heated. The moisture is acquired when moisture in the air is condensed on a surface of the cooling plate 29. The heat generated on the surfaces, which are on the side of the heat sink 28, of the Peltier elements 27 are transmitted to the heat sink 28 and are cooled by the wind from the blower fan 20. The heat sink 28 is made from aluminum having high thermal conductivity. In order to release heat to the passing air efficiently, an area in contact with the passing air is preferably large. Thus, for example, a plurality of groove parts may be included in a wind sending direction on a wall surface, which is on the side of the blower fan 20, of the heat sink 28. Also, the heat sink 28 has a shape in which a channel in a lower stream is narrowed down. As described in the first embodiment, it becomes possible to cause a reaction between generated active species and a charged fine water droplet effectively. Also, when an operation to heat the indoor air is performed in the air conditioner 15, heat-release from the heat sink 28 also contributes to heating of the indoor air. Thus, this is efficient.

Also, for generation of moisture during an operation of cooling or dehumidifying the indoor air, a method of using moisture accumulated in the dew receiving tray 23 can be also used in addition to the above method. This is because a temperature of the heat exchanger 21 becomes lower than an indoor temperature when the indoor air is cooled or dehumidified. Thus, moisture of the indoor air is condensed on the surface of the heat exchanger 21 and the condensed moisture is accumulated in the dew receiving tray 23 on a lower side of the heat exchanger 21. Also, when the indoor air is dehumidified, there is a case where an operation of warming the air passing through the heat exchanger 21 is performed again in order not to lower the temperature of the indoor air along with an operation of lowering a temperature of the heat exchanger 21 and condensing and removing moisture in the air. In this case, power consumption may become greater compared to a cooling operation. The heat-release from the heat sink 28 has an effect to prevent the indoor air from being cooled and works efficiently during a dehumidifying operation.

The moisture acquired in the dew receiving tray 23 and the cooling plate 29 is supplied to the atomization electrode 10 including a sponge with hygroscopicity. In order to make the electric discharge stable, an earthed electrode 9 is provided around the atomization electrode 10. The atomization electrode 10 has a plurality of corners. When a high voltage of −1 to −10 kV is applied, an electric field is concentrated to the corners. Then, the Rayleigh breakup of the moisture supplied to the atomization electrode 10 is caused and the moisture becomes a charged fine water droplet. Since the plurality of corners are extended to a wind channel, the generated charged fine water droplet is efficiently supplied to a plasma generating part 3 in the lower stream by the wind and electrical attraction to plasma.

The plasma generating part 3 is provided on a wall surface on a side of the wind channel of the heat sink 28. The plasma generating part 3 has the configuration illustrated in FIG. 4 of the second embodiment. The discharging electrode is covered with Al₂O₃ and is provided in such a manner as to sandwich the plasma. The height of each of the dielectric substances 13 and 14 is around 0.1 to 1.0 mm and a distance between the electrodes is 0.1 to 0.5 mm. Since the plasma generating part 3 is heated by the heat from the heat sink 28, an ozone catalyst effect of Al₂O₃ is increased and plasma 1 can be generated in a state in which ozone is decreased dramatically. Since it is possible to generate active species such as an OH radical while controlling generation of ozone, it is possible to sterilize the passing air safely at high speed.

### Fourth Embodiment

An embodiment in which a sterilization device described with reference to FIG. 1 to FIG. 4, and FIG. 9 of the first to third embodiments is applied to a self-propelled cleaner provided in a bioclean room (hereinafter, referred to as BCR) will be described in the following. In each of the first to third embodiments, an object of processing of the sterilization device is an airborne bacterium. However, in the present embodiment, a case where an adhered bacterium on a floor is also included in the object of processing will be described.

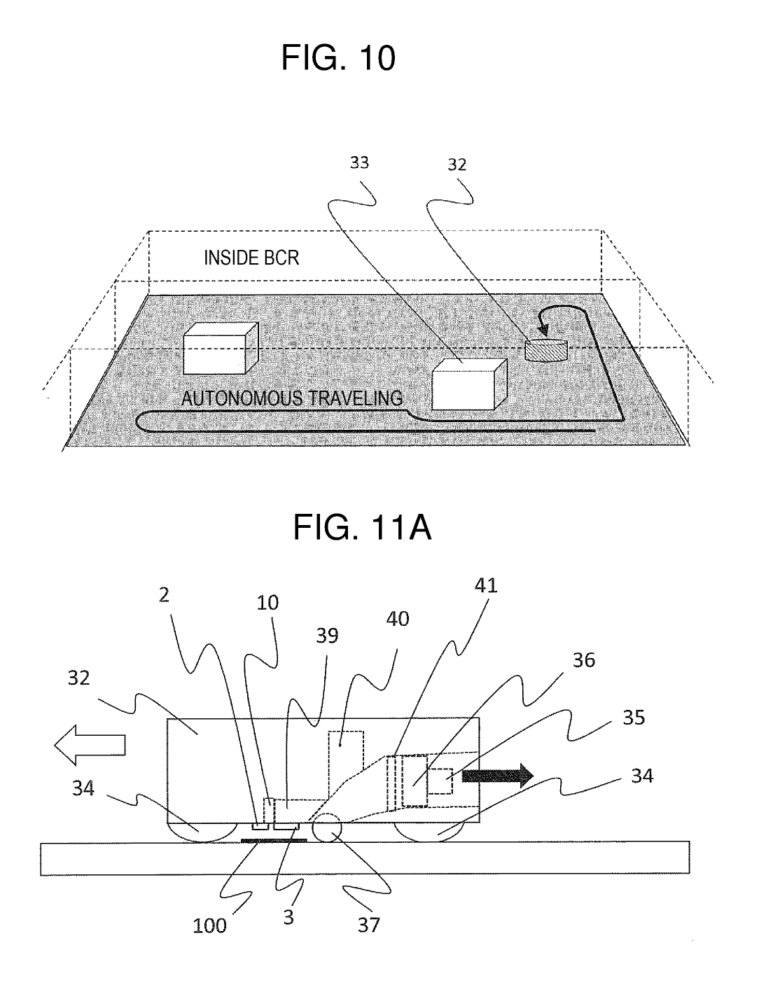

As illustrated in FIG. 10, a self-propelled cleaner 32 autonomously travels all over an object room while avoiding an obstacle 33 and removes trash such as dust on a floor based on information acquired from inside/outside the cleaner, the information being acquired from a sensor such as an infrared ray sensor or a video in a camera provided in the room, for example. When moving, the cleaner moves a wheel 34 in a lower part thereof and can perform an operation such as changing a moving direction, moving forward, or moving backward.

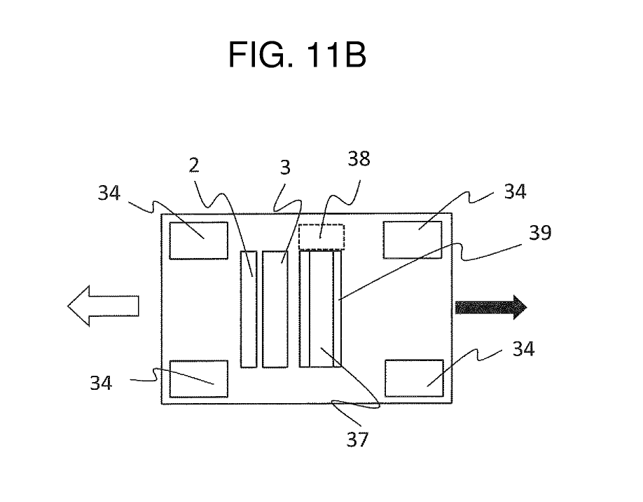

In FIG. 11(a) and FIG. 11(b), an example in which a sterilization device according to the present invention is applied to a self-propelled cleaner is illustrated. FIG. 11(a) is a side view of the self-propelled cleaner and FIG. 11(b) is a bottom view thereof. When the above device 32 is operated, a fan 36 is driven by a fan motor 35 in the cleaner and a negative pressure with respect to the atmosphere is generated in the cleaner. Also, a rotary brush 37 on a bottom surface of the cleaner is rotated by a brush motor 38 and trash is vacuumed from an inlet 39. The vacuumed trash is collected into a dust box 40 and exhaust is performed through a filter 41. A white arrow in each of FIG. 11(a) and FIG. 11(b) indicates a moving direction of the self-propelled cleaner and a black arrow indicates a direction of exhausting the air. The sterilization device according to the present invention is provided to the bottom surface of the self-propelled cleaner and is configured from a charged fine water droplet supplying part 2 and a plasma generating part 3 in this order from a side of the moving direction of the cleaner. A detail of the sterilization device is similar to that in FIG. 9 of the third embodiment. The sterilization device of the present embodiment is provided in such a manner that a discharging surface of the plasma generating part 3 becomes the lower surface.

As described in the third embodiment, when moisture supplied to the charged fine water droplet supplying part 2 is acquired by condensing moisture in the air, it is not necessary to supply water and it is convenient. For example, in a case where a Peltier element described in the third embodiment is used to generate moisture, a heat sink 28 is preferably provided on a wall surface of a channel in a vicinity of the inlet 39 and cooling is preferably performed in order to remove the heat generated in the Peltier element. As a different method of supplying moisture, for example, a tank to store water may be provided in a device. In this case, water is to be supplied to the tank arbitrarily when the water is out.

In a case of using the self-propelled cleaner, by operating and moving the sterilization device, it becomes possible to remove an adhered bacterium 100 which is on a floor and is right beneath the sterilization device or an airborne bacterium included in the air passing through the sterilization device as well as the trash on a floor. As described in the first embodiment, since a period from generation of an OH radical until deactivation thereof is short, the plasma generating part 3 is provided to be close to the surface of the floor, that is, provided about 0.5 to 5.0 mm above the floor in application to sterilization of the adhered bacterium.

Moreover, the above device 32 acquires power from a rechargeable battery in the device when operating. After moving through the BCR, the device goes back to a charging space provided in the BCR. The above device 32 may be constantly operated to keep a degree of cleanliness required for the BCR or may be operated once in a several hours or once a day.

It is possible to operate the above device 32 even when a worker is at work. However, the device may be operated when the worker is not in the BCR at night. Accordingly, it is possible to keep the room clean all the time without completely stopping the operation of the BCR for a several days for sterilization processing. Also, for removal of an adhered bacterium, the sterilization device is preferably provided on the bottom surface of the cleaner. In a case where removal of an airborne bacterium is valued more, the sterilization device may be arranged in a top surface of the cleaner or in the vicinity of an exhaust outlet. A place where the sterilization device is provided may be changed according to a purpose or may be provided in a plurality of places. Also, in the present embodiment, a self-propelled cleaner having a sterilization device has been described. However, a self-propelled sterilization device without a function of a cleaner may be used.

A sterilization device using electric discharge which device is proposed in the present invention can be used in a place, where sterilization of an airborne bacterium is necessary, such as an indoor space, a bioclean room, an aseptic room, or a culture device and can be safely used in a space where there is a human or an animal. Also, not only application to sterilization of an airborne bacterium but also application to sterilization of a bacterium adhered to a surface are possible.

### REFERENCE SIGNS LIST

1 plasma
2 charged fine water droplet supplying part
3 plasma generating part
4 high-frequency power supply
5 high-frequency electrode
6 earthed electrode
7 dielectric substance
8 moisture supplying part
9 earthed electrode
10 atomization electrode
11 high-voltage power supply
12 blower means
13 dielectric substance
14 dielectric substance
15 air conditioner
16 indoor equipment
17 outdoor equipment
18 connection pipe
19 air outlet
20 blower fan
21 heat exchanger
22 dew receiving tray
23 dew receiving tray
24 air inlet
25 air inlet
26 fan motor
27 Peltier element
28 heat sink
29 cooling plate
30 heat insulation material
31 DC power supply
32 self-propelled cleaner
33 obstacle
34 wheel
35 fan motor
36 fan
37 rotary brush
38 brush motor
39 inlet
40 dust box
41 filter
100 bacterium adhered to surface

1. A sterilization device comprising:
a charged fine water droplet supplying unit; and
a plasma generating unit,
wherein the charged fine water droplet supplying unit and the plasma generating unit are provided on a wall surface of a wind channel in order of the charged fine water droplet supplying unit and the plasma generating unit from an upper stream in a direction in which air flows,
the plasma generating unit includes a charged fine water droplet supplying part and a plasma generator,
the charged fine water droplet supplying part includes a high-voltage power supply, an earthed electrode, and an electrode to which moisture is supplied by a water feeding unit, a negative high voltage with respect to the earthed electrode being applied to the electrode supplied with moisture,
the plasma generator includes a pair of plasma generating electrodes and a high-frequency power supply, the plasma generating electrodes being covered by a dielectric substance and being provided in the same plane with the dielectric substance, and
the air is converted into plasma and emitted when voltage is applied to the plasma generating electrodes by the high-frequency power supply.

2. The sterilization device according to claim 1, wherein air including a charged fine water droplet generated in the charged fine water droplet supplying part is converted into plasma and is emitted by the plasma generating unit.

3. The sterilization device according to claim 1, wherein dielectric substances are provided to sandwich plasma in a plasma generating part.

4. The sterilization device according to claim 1, wherein a cross-sectional area of a channel of the air is decreased in a following stage of the plasma generating unit.

5. The sterilization device according to claim 1, wherein a channel of the air is connected to a second channel, a flowing direction of which is different from that of the channel, in a following stage of the plasma generating unit.

6. A sterilization device comprising:
a charged fine water droplet supplying unit;
a plasma generating unit; and
blower means,
wherein the charged fine water droplet supplying unit and the plasma generating unit are provided on a wall surface of a wind channel in this order from an upper stream in a direction in which air supplied by the blower means is sent,
the plasma generating unit includes a charged fine water droplet supplying part and a plasma generator,
the charged fine water droplet supplying part includes a high-voltage power supply, an earthed electrode, and an electrode to which moisture is supplied by a water feeding unit, a negative high voltage with respect to the earthed electrode being applied to the electrode to which moisture is supplied,
the plasma generator includes a pair of plasma generating electrodes and a high-frequency power supply, the plasma generating electrodes being covered by a dielectric substance and being provided in the same plane with the dielectric substance, and
the air is converted into plasma and emitted when voltage is applied to the plasma generating electrodes by the high-frequency power supply.

7. The sterilization device according to claim 6, wherein air including a charged fine water droplet generated in the charged fine water droplet supplying part is converted into plasma and is emitted by the plasma generating unit.

8. The sterilization device according to claim 6, wherein dielectric substances are provided to sandwich plasma in a plasma generating part.

9. The sterilization device according to claim 6, wherein a cross-sectional area of a channel of the air is decreased in a following stage of the plasma generating unit.

10. The sterilization device according to claim 6, wherein a channel of the air is connected to a second channel, a flowing direction of which is different from that of the channel, in a following stage of the plasma generating unit.
